(19) United States
(12) Patent Application Publication
(10) Pub. No.: US 2007/0173827 A1
(43) Pub. Date: Jul. 26, 2007

(54) Adjustable connector for attachment to a rod in a medical application

(75) Inventors: Matthew M. Morrison, Cordova, TN (US); John Stewart Young, Memphis, TN (US)

Correspondence Address: COATS & BENNETT, PLLC, 1400 Crescent Green, Suite 300, Cary, NC, 27518, US

(73) Assignee: SDGI Holdings, Inc.

(21) Appl. No.: 11/336,586

(22) Filed: Jan. 20, 2006

(51) Int. Cl. A61F 2/30

(52) U.S. Cl. 606/61

(57) ABSTRACT

Devices and methods for attaching a rod to an anchor. One embodiment includes attaching the rod to an anchor that extends from a vertebral member. One device may include a connector housing including a first channel sized to receive the rod and a second channel sized to receive the anchor. A contact member may be sized to at least partially extend within the second channel. Insertion of the anchor into the second channel may cause the contact member to further extend through an opening in the connector housing and into the first channel. The contact member may contact the rod and attach it to the connector housing. A fastening member may operatively connect the anchor to the connector housing. The contact member may accommodate the anchor at a variety of angular orientations within the connector housing.

### BACKGROUND

The present application is directed to devices and methods for attaching a rod to a member, and more specifically, to connectors and methods providing multiple degrees of freedom for attaching a rod to a member.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve.

A rod may be implanted to support and position a vertebral member in one or more of these regions. The rod extends along a section of the spine and is connected to the vertebral member with one or more anchors. The rod may have a curved configuration to conform to the curvature and contour of the spine.

An anchor is inserted into the vertebral member to connect the rod. Because the rod anchor cannot directly receive the anchor, a connector connects the rod to the anchor. The connector includes a first attachment that connects to the rod, and a second attachment that connects with the anchor. It is often difficult for the connector to accommodate both the rod and the anchor. The vertebral member may have a variably contoured surface that results in the rod and the anchor each being positioned at a variety of angular orientations.

Rods may be used in other medical applications, such as treatment of orthopedic traumas. In one embodiment, rods are internally situated within the patient and should be anchored with an internal fixation device. In other embodiments, rods may be externally situated and require external fixation devices to anchor and locate the rod.

### SUMMARY

The present application is directed to devices and methods for attaching a rod to an anchor. The device may include a connector housing having a first channel sized to receive the rod and a second channel sized to receive the anchor. A contact member may be sized to at least partially extend within the second channel. Insertion of the anchor into the second channel may cause the contact member to extend through an opening in the connector housing and into the first channel. The contact member may contact the rod and attach it to the connector housing. A fastening member may operatively connect the anchor to the connector housing. The contact member may accommodate the anchor at a variety of angular orientations within the connector housing.

### BRIEF DESCRIPTION OF THE DRAWINGS

FIG. 1 is a perspective view of a connector according to one embodiment.

FIG. 2 is an exploded view of a connector according to one embodiment.

FIG. 3 is a cross section view of a connector positioned between a rod and an anchor according to one embodiment.

FIG. 4 is a cross section view of a connector positioned between a rod and an anchor according to one embodiment.

FIG. 5 is a cross section view of a connector positioned between a rod and an anchor according to one embodiment.

FIG. 6 is an exploded view of a connector according to one embodiment.

FIG. 7 is a side view of a contact member according to one embodiment.

FIG. 8 is a side view of a contact member according to one embodiment.

### DETAILED DESCRIPTION

The present application is directed to connectors and methods of connecting a rod with an anchor. In one embodiment, the connector includes a housing that extends between the rod and the anchor. The connector may include adjustable elements that accommodate the anchor and rod at a variety of different angular orientations. In one embodiment, the connectors and methods are useful for attaching a rod to a vertebral member. The connectors and methods may also be used in other embodiments, such as treatment of orthopedic trauma applications. The connectors and methods may be applicable internally within the body, or for external fixation.

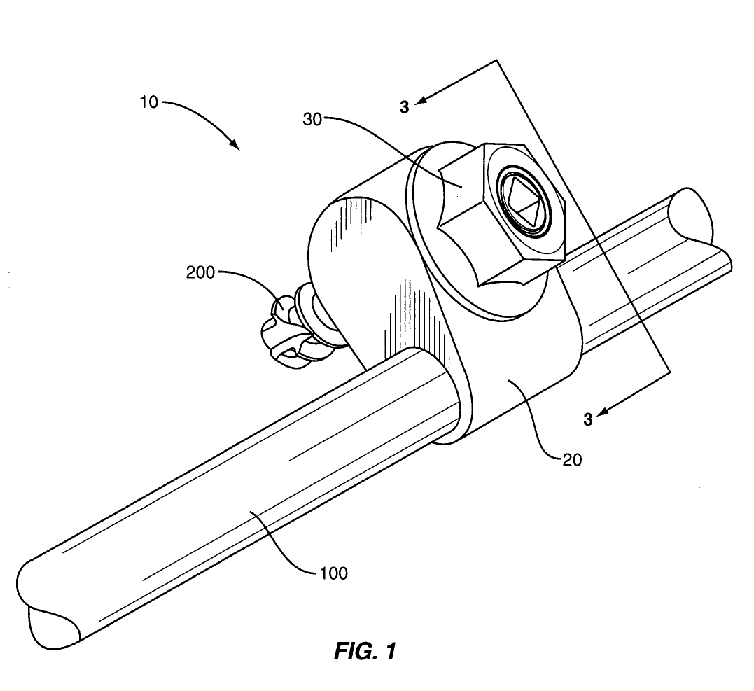

One embodiment of a connector 10 is illustrated in FIG. 1. Connector 10 includes a housing 20 sized to extend between a rod 100 and an anchor 200. A first channel extends through the housing 20 to receive the rod 100. A second channel extends through the housing to receive the anchor 200 at a point adjacent to the rod 100. A fastening member 30 is operatively connected to the anchor 200 to attach the rod 100 to the housing 20.

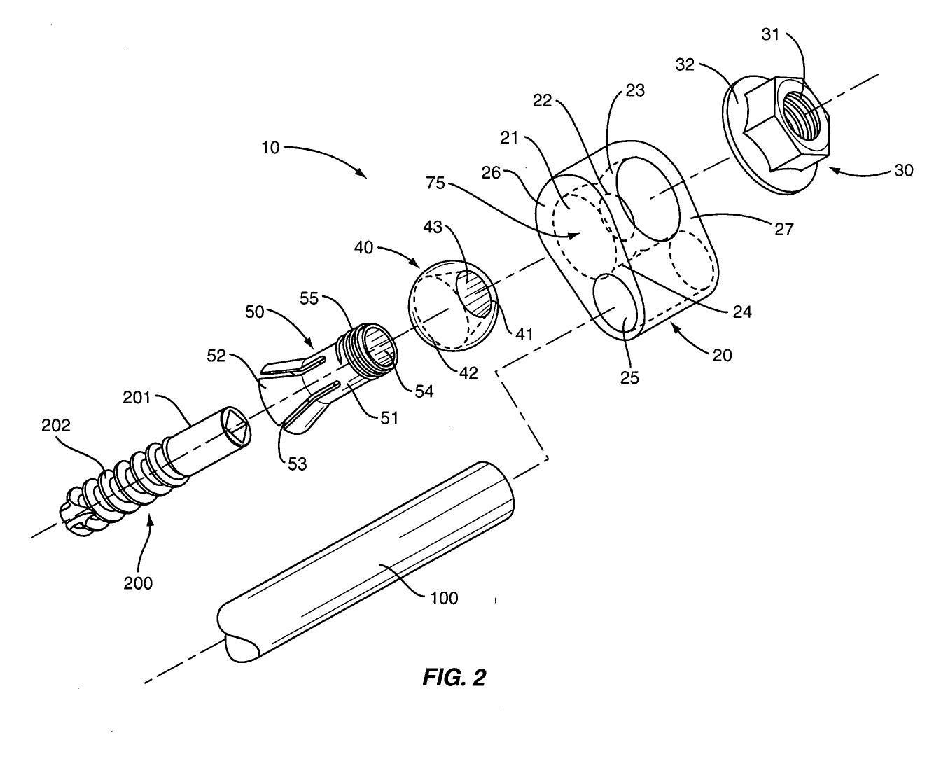

FIG. 2 illustrates an exploded view of one embodiment of a connector 10. In this embodiment, connector 10 includes a housing 20, fastening member 30, contact member 40, and a collet 50. The housing 20 is sized to connect the rod 100 and anchor 200. In one embodiment, a first channel 25 extends through the housing 20 and is sized to receive the rod 100. In this embodiment, channel 25 includes a circular cross-sectional shape sized to receive the rod 100. Channel 25 may include other shapes each sized to receive the rod 100. In one embodiment as illustrated in FIG. 1, the cross-sectional shape of the channel 25 is substantially the same as the rod 100. In other embodiments, the channel 25 and rod 100 include different cross-sectional shapes. The size of the channel 25 relative to the rod 100 allows the housing 20 to translate along the length of the rod 100. The size also allows the housing 20 to be positioned at a variety of angular orientations relative to the rod 100. In one embodiment as illustrated in FIG. 2, channel 25 is formed within an interior of the housing 20. In another embodiment, channel 25 is formed on an outer edge of the housing 20 and includes an open side.

Housing 20 further includes a second channel 75 for attachment with the anchor 200. In one embodiment, the second channel 75 is substantially transverse to the orientation of the first channel 25 that houses the rod 100. In the embodiment illustrated in FIG. 2, second channel 75 includes a first cavity 21 and a second cavity 23. The first cavity 21 extends inward from a first face 26 of the housing 20, and the second cavity 23 extends inward from a second face 27. In one embodiment, cavities 21, 23 include a larger width at the faces 26, 27 and reduce to a smaller width at an interior section of the housing 20. In one embodiment as illustrated in FIG. 2, cavities 21, 23 include curved surfaces. In another embodiment, cavities 21, 23 include a conical shape with substantially straight surfaces. Cavities 21, 23 may include the same shape and size, or may include different shapes and sizes.

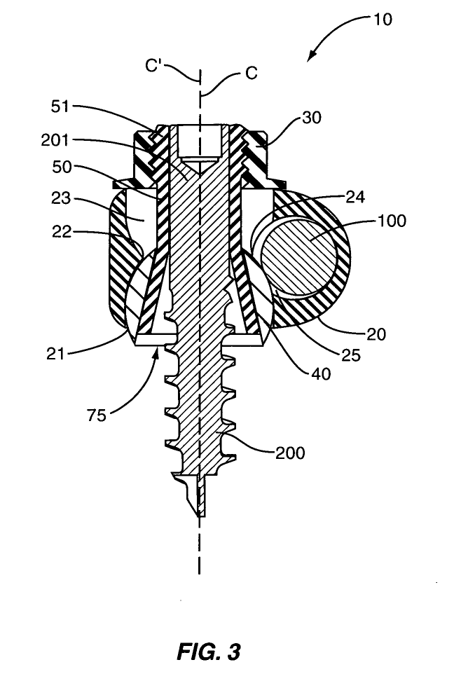

In one embodiment, the cavities 21, 23 extend into the housing and connect together at a neck 22 such as illustrated in the embodiments of FIGS. 2 and 3. In one embodiment, the cavities 21, 23 are distanced apart and the neck 22 includes a length to extend between and connect the cavities 21, 23. In one embodiment, neck 22 is positioned within a center of the housing 20 with each of the cavities 21, 23 having substantially the same depth. In another embodiment, neck 22 is positioned in closer proximity to one of the first and second faces 26, 27.

In one embodiment, the first and second channels 25, 75 are positioned within the housing 20 in an overlapping substantially transverse configuration. This overlap forms an opening 24 sized for a portion of the contact member 40 to extend through (as will be explained in detail below). Opening 24 may be positioned at a variety of depths within the housing 20 between the first and second faces 26, 27. In one embodiment, the first cavity 21 extends into the channel 25 forming an opening 24.

Contact member 40 is sized and positioned within the first cavity 21. A first edge 41 may be urged towards the neck 22, and a second edge 42 may be urged towards the first face 26. In one embodiment, member 40 is positioned completely within the first cavity 21 (i.e., the length of the member 40 is less than a length of the cavity 21 causing the second edge 42 of the contact member 40 to be positioned inward of the first face 26). In another embodiment, the member 40 extends outward from the first cavity 21.

In one embodiment, the contact member 40 includes a partially convex shape. This may include the entirety of the contact member 40 being convex, or a limited section being convex. One embodiment features the contact member 40 having a spherical shape. In one embodiment as illustrated in FIG. 3, contact member 40 includes a mostly-spherical or ellipsoidal shape. An aperture 43 extends through the contact member 40 from the first edge 41 to the second edge 42. In one embodiment, aperture 43 includes a tapered shape with a smaller width at the first edge 41 that increases to a larger width at the second edge 42. The taper may be straight, or may be convex.

In one embodiment, a collet 50 extends over the anchor 200 and within the aperture 43 of the contact member 40. In one embodiment as illustrated in FIG. 2, collet 50 includes a first section 51 with a substantially constant width and a second flared section 52. The first section 51 is also sized to fit through the neck 22 within the housing 20. Threads 55 may be positioned on an outer edge of the first section 51 to engage with the fastening member 30. In one embodiment, the first section 51 includes slits that extend substantially perpendicular to the threads 55.

The second section 52 includes a flared configuration that enlarges outward away from the first section 51. The flared configuration may be formed from substantially straight or curved sidewalls. Slits 53 may extend through a portion or entirety of the second section 52 to provide flexibility. Slits 53 may further extend into the first section 51. An interior section 54 extends through the collet 50 and is sized to receive the anchor 200.

In one embodiment in a vertebral application, a variety of different anchors 200 may attach the rod 100 to the vertebral member. In one embodiment, anchor 200 includes a non-threaded first section 201 and a threaded second section 202. The non-threaded section 201 may extend along the length of the anchor 200 a variety of distances. In one embodiment as illustrated in FIG. 2, section 201 includes a substantially circular cross-sectional shape. The threaded section 202 is configured to bite into the vertebral member and may terminate at a tip.

Rod 100 may have a variety of lengths. In one embodiment used in a vertebral application, rod 100 includes a length to extend along the spine and support one or more vertebral members. Rod 100 may include a variety of cross-sectional shapes, sizes, and materials depending upon the context of use. In one embodiment as illustrated in FIG. 2, rod 100 includes a circular cross-sectional shape.

Fastening member 30 includes a threaded aperture 31 that attaches with the threads 55 on the collet 50. In one embodiment, the fastening member 30 is positioned on the second face 27 of the housing 20 when connected with the collet 50. In one embodiment, a flange 32 extends radially outward from the aperture 31 and includes a width to extend over the second cavity 23. In one embodiment, fastening member 30 is a nut having a polygonal head with a threaded interior.

FIG. 3 illustrates one embodiment of an assembled connector 10 that connects the rod 100 and anchor 200. The anchor 200 is positioned within the collet 50 with the anchor first section 201 extending into the collet first section 51. In this embodiment, a top edge of the anchor 200 is substantially aligned with a top edge of the collet 50. The collet 50 and inserted anchor 200 are sized to fit within cavities 21, 23 and the neck 22. The collet 50 extends outward beyond the housing 20 and is secured in position by the fastening member 30.

The collet 50 and inserted anchor 200 are further positioned within the aperture 43 of the contact member 40. When inserted into the first cavity 21, the contact member 40 seats against the interior sidewalls of the first cavity 21. This positioning further causes the contact member 40 to extend through the opening 24 and into contact with the rod 100. This contact between the contact member 40 maintains the rod 100 fixedly connected to the housing 20.

One embodiment features a vertebral application. The method of connecting the rod 100 and anchor 200 comprises initially mounting the anchor 200 to a vertebral member. The anchor 200 is mounted at a location in proximity to the vertebral rod 100. The threaded section 202 seats within the vertebral member with the non-threaded section 201 extending outward from the vertebral member. The rod 100 is inserted into the channel 25 within the housing 20, and the housing 20 is moved along the length of the rod 100 to a point in proximity to the anchor 200.

The collet 50 is connected to the anchor 200 by inserting the first section 201 that extends out from the vertebral member into the interior section 54 of the collet 50. In one embodiment, collet 50 is inserted onto the anchor 200 with the top end of the first section 201 being substantially aligned with a top edge of the collet first section 51. In another embodiment, collet 50 is inserted over the anchor 200 to a lesser extent. The contact member 40 is then inserted onto the collet 50 and anchor 200. Specifically, aperture 43 of the contact member 40 is placed over the collet first section 51 and anchor 200. In one embodiment, collet 50 can be inserted into the aperture 43 until the second edge 42 contacts the flared second section 52.

The collet 50 and anchor 200 are then inserted into the housing 20. Specifically, the collet first section 51 is inserted through the second channel 75 including the first cavity 21, neck 22, and second cavity 23 until the first section 51 extends outward above the second face 27 of the housing 20. Fastening member 30 is then connected to the collet first section 51. In one embodiment, the threaded aperture 31 of the fastening member 30 engages the threads 55 on the first section 51 of the collet 50.

As the collet first section 51 is inserted through the housing 20, the contact member 40 is moved into the interior of the first cavity 21. This movement may be caused by manually inserting the first section 51 through the housing 20, and/or by threadingly engaging the fastening member 30 onto the collet 50. In another embodiment, a ratcheting mechanism moves the collet 50 into the channel 75. This embodiment may lock the collet 50 within the channel 75 and include a release clip to loosen the collet 50. Movement of the contact member 40 into the first cavity 21 causes the contact member 40 to move through the opening 24 and contact the rod 100. The contact member 40 may move into the first cavity 21 until it seats against the inner surfaces of the cavity 21, until it is prevented from further movement because of contact with the rod 100, or both. In one embodiment as illustrated in FIG. 3, contact between the contact member 40 and rod 100 moves the rod 100 against the wall of the channel 25 thus locking the rod 100 to the housing 20.

In one embodiment, the flared second section 52 compresses inward as the collet 50 moves through the contact member 40. This compression may further lock the collet 50 to the contact member 40, and lock the collet 50 to the anchor 200.

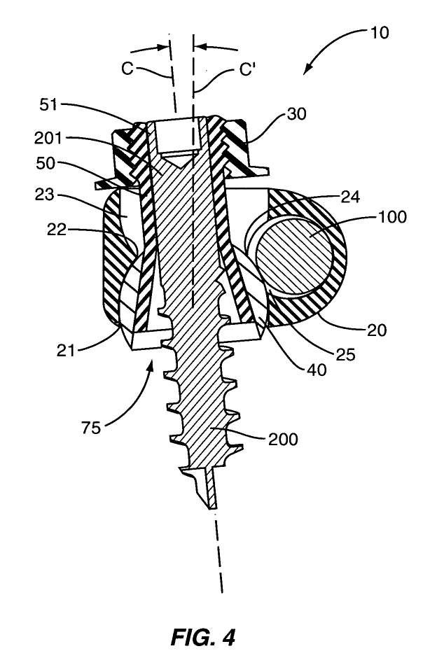

In one embodiment as illustrated in FIG. 3, a centerline C of the anchor 200 is substantially aligned with a centerline C′ that extends through the second channel 75 including the cavities 21, 23 and neck 22. The shapes of the cavities 21, 23 accommodate movement of the anchor 200 within the housing 20. As illustrated in FIG. 4, anchor 200 has been pivoted such that the anchor centerline C is offset from the centerline C′. During this pivoting movement, the relative positions of the anchor 100, contact member 40, and collet 50 remain substantially the same. However, the motion causes the collet 50, contact member 40, and anchor 200 to pivotally move within the first cavity 21. The curved shape of the contact member 40 may slide along the surface of the rod 100 and the first cavity 21.

In one embodiment as illustrated in FIG. 4, the connector 10 accommodates multiple degrees of freedom. Relative to the rod 100, the housing 20 may rotate about the rod 100 as it is positioned within the channel 25, and also move along the length of the rod 100. Relative to the anchor 200, the housing 20 may rotate about the anchor 200, and may also be angled within two planes. In an embodiment with the anchor 200 extending substantially outward in a posterior direction from the vertebral member, housing 20 can be angulated within the sagittal and axial planes.

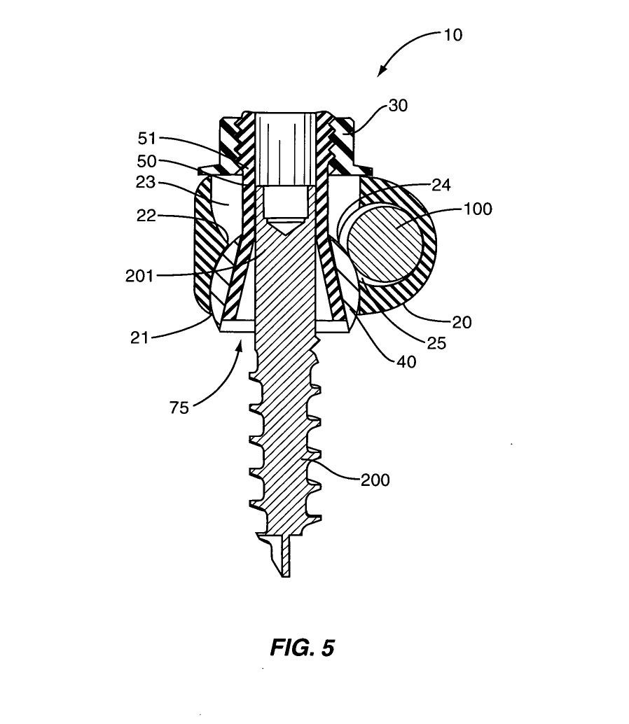

In one embodiment as illustrated in FIGS. 3 and 4, the collet 50 is inserted on the anchor 200 with a top edge of the anchor 200 being substantially aligned with a top edge of the collet 50. In another embodiment as illustrated in FIG. 5, collet 50 is inserted a lesser amount into the anchor 200. In this embodiment, the anchor 200 is inserted into the collet 50 with a top edge of the anchor 200 being slightly within the first section 51 of the collet 50.

In one embodiment, the collet 50 is maintained on the anchor 200 by a compressive force applied by the collet 50. In one embodiment, the collet 50 is constructed of a flexible material that stretches during insertion of the anchor 200 and applies a holding force. In another embodiment, insertion of the contact member 40 onto the flared section 52 of the collet 50 causes the flared section 52 to move inward and exert a compressive force onto the anchor 200.

In one embodiment, contact member 40 is constructed of a rigid material. In another embodiment, contact member 40 is constructed of a resilient material that elastically deforms when locked against the rod 100.

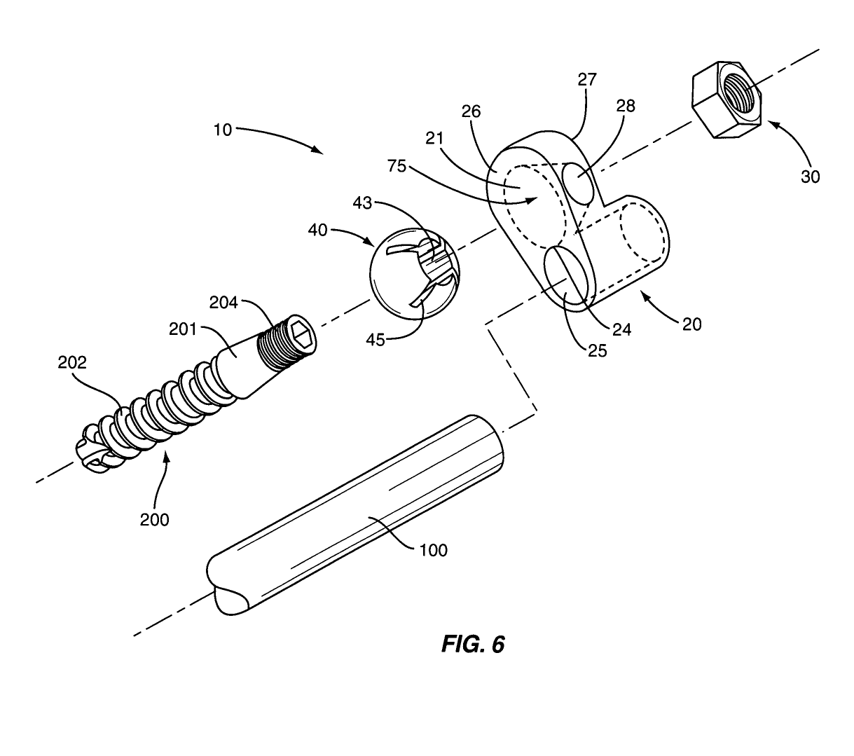

FIG. 6 illustrates another embodiment having an anchor 200 that connects directly with the fastening member 30. Specifically, anchor 200 includes a threaded end 204 that engages a threaded fastening member 30. The housing 20 includes a first channel 25 to receive the rod 100, and a second channel 75 configured to receive the contact member 40. The second channel 75 has a variable shape that is wider at the first face 26 and narrower at the second face 27. The second channel 75 terminates at an opening 28 at the second face 27. In one embodiment, opening 28 includes a greater width than a width of the threaded end 204 of the anchor 200 to provide for pivoting movement of the anchor 200 relative to the housing 20.

Contact member 40 mounts onto the anchor 200. In one embodiment, an aperture 43 having a substantially constant width extends through the contact member 40. Slits 45 may be formed in the contact member 40. In one embodiment, the first end 201 of the anchor 200 includes a tapered width that grows towards the threaded section 202.

Use of the embodiment of FIG. 6 comprises inserting the threaded section 204 of the anchor 200 through the contact member 40. The anchor 200 is inserted through the second channel 75 with the threaded section extending through the opening 28 to receive the fastening member 30. Movement of the anchor 200 through the opening 28 causes the contact member 40 to slide past the threaded section 204 and onto the tapered first section 201. This movement may cause the aperture 43 to expand along the slits 45 as the contact member 40 moves along the tapered first section 201. The expansion of the contact member 40 and/or movement of the contact member 40 onto the tapered section 201 mounts the contact member 40 to the anchor 20. Contact member 40 further moves into the cavity 21 and into the opening 24 to contact and lock the rod 100 to the housing 20.

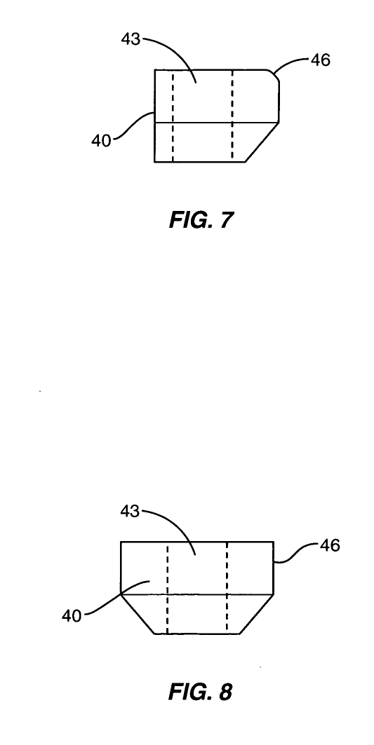

Contact member 40 includes a variety of shapes. In the embodiment of FIG. 2, member 40 includes a substantially semi-spherical shape with a first edge 41 being curved and the second end 42 being truncated. In another embodiment, member 40 includes a spherical shape. Contact member 40 may be symmetrical about the aperture 43 such as the embodiments illustrated in FIG. 2. Contact member 40 may also be non-symmetrical. FIG. 7 illustrates one embodiment having a contact surface 46 that is non-symmetrical relative to the aperture 43. In one embodiment, contact member 40 includes a curved surface that slides against the rod and/or second channel to accommodate the pivoting movement of the anchor 200. Other embodiments may include a contact surface 46 that is substantially straight such as the embodiment illustrated in FIG. 8.

The first and second channels 25, 75 may extend through the housing 20 at a variety of different positions. In one embodiment, the first channel 25 that receives the rod 100 is substantially perpendicular to the second channel 75 that receives the anchor 200. In one embodiment, a second opening (not illustrated) extends between the first and second channels 25, 75. In one embodiment, the openings are spaced apart within the housing 20. In one embodiment, the first opening 24 is positioned within the first cavity 21 and a second opening is positioned within the second cavity 23. In one embodiment, multiple openings are positioned within a single cavity 21, 23.

In one embodiment, channel 75 is symmetrical such that the connector can be assembled from either direction. In one embodiment, channel 75 includes two symmetrically formed convex surfaces with substantially equivalent internal geometric features.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. An assembly to connect a rod to an anchor comprising:
a housing having a first channel to receive the rod and a second channel to receive the anchor, the first channel extending into the second channel and forming an opening therebetween;
a contact member having an aperture to fit over the anchor, the contact member having an exterior surface that extends into the first channel and contacts the rod when the anchor is positioned within the second channel; and
a fastening member operatively connectable to the anchor to maintain the anchor within the second channel.

2. The assembly of claim 1, wherein the first channel and the second channel are substantially perpendicular.

3. The assembly of claim 1, wherein the exterior surface of the contact member includes a convex ellipsoid segment surface.

4. The assembly of claim 1, wherein the second channel comprises a first cavity and a second cavity, each of the cavities having an enlarged outer width that decreases to a narrow inner width.

5. The assembly of claim 1, further comprising a second opening extending between the first channel and the second channel, the second opening being spaced apart from the opening.

6. The assembly of claim 1, wherein the contact member is substantially semi-spherical.

7. The assembly of claim 1, further comprising a collet positioned between the contact member and the anchor, the collet having a fastening surface that attaches with the fastening member.

8. The assembly of claim 1, wherein the contact member extends outward from the second channel when the fastening member is mounted to the anchor.

9. An assembly to connect a rod to an anchor comprising:
a housing having a first channel to receive the rod and a second channel to receive the anchor, the second channel having a varying width section that extends between an enlarged first section and a reduced second section, the first channel and the second channel forming an opening that extends therebetween,;
a fastening member operatively connectable to the anchor to maintain the anchor within the second channel; and
a contact member having a curved exterior surface, the contact member being attachable to the anchor and sized to fit within the second channel and extend through the opening and into the first channel to contact and maintain the rod when the fastening member is attached to the anchor.

10. The assembly of claim 9, wherein the opening extends between the first channel and the varying width section of the second channel.

11. The assembly of claim 9, wherein the second channel comprises a second varying width section, the varying width section extending inward into the housing from a first face of the housing and the second varying width section extending inward into the housing from a second face of the housing with the reduced second section being aligned within an interior of the housing and away from the first and second faces.

12. The assembly of claim 9, wherein the contact member includes a substantially ellipsoidal shape.

13. The assembly of claim 9, wherein the contact member includes an aperture sized to receive the anchor.

14. The assembly of claim 9, further comprising a collet positioned between the contact member and the anchor, the collet having a fastening surface that fastens with the fastening member.

15. An assembly to connect a rod to an anchor comprising:
a housing having a first channel to receive the rod and a second channel to receive the anchor, the second channel comprising first and second cavities each having a varying width extending between a wide exterior end and a narrow interior end, the first channel extending into the second channel and forming an opening therebetween;
a fastening member operatively connectable to the anchor to maintain the anchor within the second channel; and
a contact member having a curved exterior surface, the contact member configured to be held within the first cavity by the anchor and extend through the opening and into the first channel to contact and lock the rod to the housing.

16. The assembly of claim 15, further comprising a neck section sized to receive the anchor and extending between the narrow interior ends of the first and second cavities.

17. The assembly of claim 15, wherein the curved exterior surface of the contact member complements the narrow interior end of the first cavity.

18. The assembly of claim 15, wherein the first channel is substantially perpendicular to the second channel.

19. The assembly of claim 15, wherein the contact member extends outward from the second channel when held within the first cavity by the anchor.

20. The assembly of claim 15, further comprising an aperture that extends through the contact member and sized to receive a portion of the anchor.

21. An assembly to connect a rod to an anchor comprising:
a housing having a first channel to receive the rod and a second channel to receive the anchor, the second channel comprising first and second cavities each having extending between a wide exterior end and a narrow interior end, the first cavity and the first channel having an opening that extends therebetween;
a collet sized to fit over a portion of the anchor and sized to extend through the second channel;
a fastening member operatively connectable to the collet; and
a contact member having a curved exterior surface and being sized to extend into the first cavity, the contact member having an aperture to extend over the collet, the contact member sized to extend through the opening and into the first channel to contact the rod.

22. The assembly of claim 21, wherein the collet comprises a first cylindrical section having a substantially constant width and a second flared section that flares outward away from the first section.

23. The assembly of claim 22, wherein the aperture within the contact member includes a width greater than the first cylindrical section and less than the second flared section.

24. An assembly to connect a rod to an anchor comprising:
a housing having a first channel to receive the rod and a second channel to receive the anchor that is asymmetrically bias to provide greater angulation on one side, the first channel extending into the second channel and forming an opening therebetween;
a contact member having an aperture to fit over the anchor, the contact member having an exterior surface that extends into the first channel and contacts the rod when the anchor is positioned within the second channel; and
a fastening member operatively connectable to the anchor to main the anchor within the second channel.

25. A method of connecting a rod with an anchor that extends from a member, the method comprising the steps of:
inserting the rod through a first channel of a connector housing;
attaching a contact member to the anchor and inserting the anchor through a second channel of the connector housing; and
connecting the anchor to the connector housing and causing the contact member to extend through an opening between the first and second channels to contact the rod and lock the rod within the first channel.

26. The method of claim 25, further comprising sliding a curved surface of the contact member against a curved wall of the second channel and positioning the connector housing relative to the anchor.
